(19) United States
(12) Patent Application Publication
(10) Pub. No.: US 2026/0347919 A1
(43) Pub. Date: Nov. 5, 2026

(54) Hybrid Algorithmic-Quantum Logic Framework for Multilayer Biomarker Analysis and Diagnostic Indexing Using HAQ Diagnostic Index

(71) Applicant: Pradheep Chhalliyil, Chesterfield, MO (US)

(72) Inventor: Pradheep Chhalliyil, Chesterfield, MO (US)

(21) Appl. No.: 19/260,633

(22) Filed: Jul. 7, 2025

Related U.S. Application Data
(63) Continuation-in-part of application No. 18/592,758, filed on Mar. 1, 2024.

(30) Foreign Application Priority Data
May 4, 2025 (WO) PCT/US25/27656

(51) Int. Cl. C12Q 1/6851; G01N 33/53

(52) U.S. Cl. CPC C12Q 1/6851; G01N 33/5308; G01N 2800/50; G01N 2800/52

(57) ABSTRACT

A multilayer diagnostic system is disclosed for early detection of disease risk, molecular imbalance, and physiological stress using non-invasive and minimally invasive sampling of multiple biofluids, including but not limited to blood, urine, saliva, stool, and sweat. The invention introduces Reserve-Excretion Ratios (RER™) to evaluate antioxidant retention, oxidative stress clearance, and detoxification efficiency across systemic and excretory fluids. Analytes include polyphenols, malondialdehyde (MDA), and cell-free nucleic acids (cfDNA, cfRNA, cf-mtDNA). A hybrid computational framework—combining machine learning and quantum-informed logic—generates a composite Quantum Diagnostic Score (QDS) and HAQ Diagnostic Index™. Chrono-synchronized fasting protocols (12, 24, 36 hours) capture dynamic biomolecular fluctuations. Experimental data confirm that chemically optimized phase-separation outperforms physical methods in cf-mtDNA extraction. The platform supports personalized, predictive diagnostics and longitudinal aging prediction across clinical, wellness, and at-home applications.

### CROSS-REFERENCE TO RELATED APPLICATIONS

This application claims the benefit of and is a continuation-in-part of U.S. Non-Provisional patent application Ser. No. 18/592,758 titled “One or Multiple-Steps Extraction of Nucleic Acids and other Biomolecules using a Cloudy Precipitated Solution” filed on Mar. 1, 2024, and U.S. Non-Provisional patent application Ser. No. 18/885,041 titled “Method and Device for the Purification, Isolation of Biomolecules” filed on Sep. 13, 2024. This application also incorporates disclosures from International PCT Application No. PCT/US25/27656 and U.S. Provisional Application No. 63/811,588.

### FIELD OF THE INVENTION

The present invention generally relates to the fields of predictive diagnostics, preventive healthcare, systems biology, and personalized medicine. More specifically, the invention discloses an integrated, multi-biomarker diagnostic platform for the early detection, risk stratification, and longitudinal monitoring of physiological imbalances, disease susceptibility, biological aging, organ stress, and therapeutic responsiveness. The platform analyzes biomolecular reserve and excretion dynamics across a range of biological samples both invasive and non-invasive using time-synchronized collection and hybrid computational interpretation.

In various embodiments, the invention utilizes biological materials derived from human or animal subjects, including but not limited to, invasive samples such as blood, tissue biopsies, cerebrospinal fluid (CSF), or other clinically obtained fluids; and non-invasive or minimally invasive specimens such as urine, saliva, stool, sweat, exhaled breath condensate, skin swabs, vaginal secretions, semen, and other secretions or exfoliates. These sample types are analyzed to quantify a wide range of molecular biomarkers, which may include, but are not limited to, antioxidant compounds (e.g., polyphenols), oxidative stress byproducts (e.g., malondialdehyde), organelle-derived nucleic acids (e.g., cf-mitochondrial DNA, cfRNA), cytokines, microbial and parasitic nucleic acids, metabolic toxins, immune mediators, and epigenetic indicators.

A distinctive and non-limiting feature of the invention includes the comparative profiling of biomarker distributions under varied physiological states, including but not limited to fasting, fed, resting, stressed, or circadian-aligned conditions. This dynamic profiling enables the detection of latent metabolic overload, detoxification inefficiencies, oxidative imbalance, and preclinical organelle stress factors that are often undetectable through conventional diagnostic methods. The sampling protocol may incorporate single or multiple temporally spaced collection intervals, including but not limited to 12-hour, 24-hour, 36-hour, or 48-hour timepoints. Additional intervals (e.g., 10, 18, 30, or 72 hours) may also be employed based on clinical requirements, biological rhythms, or operational feasibility. This flexible temporal design enhances diagnostic resolution by capturing time-dependent biomarker fluctuations across systemic and excretory compartments.

The invention further comprises a hybrid diagnostic interpretation framework that combines deterministic methods such as biomarker ratios and threshold-based analysis with non-deterministic, quantum-inspired reasoning models capable of handling multistate biological variability, uncertainty, and personalized physiological patterns. This hybrid logic system generates predictive scores, health trajectories, risk alerts, and individualized therapeutic or lifestyle recommendations.

The system is applicable across a broad spectrum of use cases, including but not limited to clinical diagnostics, digital health platforms, wellness monitoring, preventive medicine, telehealth, and at-home testing environments. It is modular, extensible, and configurable for real-time, retrospective, or longitudinal health tracking across varied populations, sample types, and biomarker categories. Accordingly, the invention provides a holistic, scalable, and adaptive solution for integrative health diagnostics leveraging multi-compartment biomolecular insights to inform precision healthcare, early disease interception, personalized treatment, and long-term resilience monitoring in both clinical and non-clinical settings.

### BACKGROUND OF THE INVENTION

Traditional diagnostic systems predominantly rely on static, episodic, and compartment-limited measurements of biomarkers often restricted to invasive samples such as blood or tissue. These conventional models are reactive in nature, typically detecting disease only after the manifestation of overt symptoms, and lack the resolution to capture early molecular shifts. Moreover, they frequently overlook dynamic physiological variables such as inter-compartmental biomolecule distribution (e.g., between blood, urine, saliva, stool, and sweat), circadian rhythm alignment, or biomarker fluctuations in response to fasting and metabolic transitions. As a result, traditional diagnostics fail to offer a real-time, integrative, or system-wide view of a subject's molecular health status.

A significant limitation of existing diagnostic approaches is the absence of molecular clearance tracking and excretion profiling features that form the foundation of the present invention's novelty. Current tools do not assess how bioactive compounds, toxins, or damaged biomolecules are mobilized, processed, or eliminated through excretory pathways. Prior art often evaluates oxidative stress indicators such as malondialdehyde (MDA), or antioxidant levels such as polyphenols, in isolation without accounting for reserve-to-clearance dynamics or correlated gene expression patterns.

In contrast, the present invention introduces the Reserve Excretion Ratio (RER™), a novel diagnostic model that quantifies the relationship between retained biomolecules in systemic compartments and their excretion through biological fluids. By dynamically analyzing multiple biological matrices including urine, saliva, stool, sweat, and blood the system enables interpretation of how molecular components such as cell-free mitochondrial DNA (cf-mtDNA) and cell-free RNA (cfRNA) are distributed, retained, or cleared, serving as early indicators of organelle stress, cellular damage, and oxidative overload. The same principle is extended to antioxidant reserves (e.g., polyphenols, phytochemicals) and oxidative damage markers (e.g., lipid peroxidation byproducts such as MDA), allowing cross-compartment analysis of redox imbalances.

Specifically, existing diagnostic frameworks fail to:
Detect pre-symptomatic molecular imbalances indicative of early disease risk, systemic dysfunction, or organelle distress.
Leverage artificial intelligence (AI), machine learning (ML), or quantum-informed models for contextualizing complex biomolecular datasets.
Provide temporally resolved, fasting-responsive, or multi-compartment diagnostic outputs based on real-time physiological dynamics.

There is a clear and unmet need for a next-generation diagnostic framework that is:
Integrated, combining physical, chemical, biological, and computational insights.
Fluid-responsive, enabling interpretation of biomarker shifts across multiple bodily fluids and sample types.
Organelle-aware, capable of detecting mitochondrial or subcellular damage using cf-mtDNA and cfRNA as early indicators.
Algorithmically advanced, blending deterministic machine learning with non-binary, quantum-inspired logic for pattern recognition, uncertainty modeling, and multi-state decision frameworks.
   Unlike conventional ML-based diagnostic algorithms that rely on rigid binary logic (“yes/no” or “present/absent”), the present invention introduces a Hybrid Computational Model that synergizes data-driven prediction (via machine learning) with quantum logic principles. Quantum logic supports modeling of ambiguous, fuzzy, and probabilistic outcomes—reflecting the true complexity of human physiology. This includes—but is not limited to—applications involving multi-biofluid diagnostics, redox modeling, aging prediction, and the detection of complex physiological states influenced by factors such as diet, emotional stress, physical exertion, fasting duration, circadian misalignment, and mindfulness or behavioral adaptation. The system further enables personalized, predictive, and preventive diagnostic insights particularly in conditions involving mitochondrial dysfunction, metabolic inefficiency, immune imbalance, and molecular aging.

### SUMMARY OF THE INVENTION

The present invention provides a comprehensive, multi-dimensional diagnostic system designed for the early detection, interpretation, and management of physiological stress, disease risk, and aging trajectories. It integrates physical, chemical, biological, mathematical, and philosophical components into a unified five-layer diagnostic framework. This model captures real-time physiological dynamics by analyzing biomarker fluctuations across invasive and non-invasive biological samples, collected from multiple compartments and at multiple time points.

In various embodiments, the five diagnostic layers include:
Physical Layer: Evaluates organ and tissue status based on symptomatology and sample-derived physiological data, correlating clinical observations with molecular signatures.
Chemical Layer: Quantifies internal biochemical conditions by measuring antioxidants, oxidative byproducts, toxins, and other small molecules reflective of redox and detox balance.
Biological Layer: Assesses cellular and organelle integrity through nucleic acid profiling, particularly cell-free DNA (cfDNA), cell-free RNA (cfRNA), and mitochondrial DNA (cf-mtDNA), which are early indicators of cellular stress and dysfunction.
Mathematical Layer: Computes diagnostic indices such as the Redox Reserve-Excretion Ratio (RER™) to quantify the body's capacity to retain, eliminate, and balance key biomolecules providing a structured interpretation of metabolic efficiency, toxin clearance, and oxidative burden.
Philosophical Layer: Integrates the multilayered biomarker data to recommend individualized interventions spanning diet, lifestyle, fasting, mindfulness, and other behavioral changes aimed at restoring health resilience and long-term system equilibrium.

A central innovation of the invention is the introduction of the Redox Reserve Excretion Ratio (RER™), which compares biomarker concentrations retained in systemic compartments (e.g., blood) to those excreted through accessible fluids (e.g., urine, saliva, stool, and sweat). This dual-compartment framework offers a refined diagnostic lens into antioxidant buffering capacity, metabolic overload, redox imbalance, and inefficiencies in molecular detoxification. High or low RER™ values may indicate oxidative stress, nutrient depletion, toxin retention, or pathological dysregulation even in asymptomatic individuals.

The invention further discloses a novel, chrono-synchronized diagnostic protocol involving time-staggered, multi-biofluid sampling under defined fasting conditions. In certain embodiments, samples are collected at two or more temporally spaced intervals, including, but not limited to, 12-hour overnight fasting, 24-hour prolonged fasting, and optionally 36-hour extended fasting under physician supervision. To accommodate biological variability and ensure diagnostic flexibility, other intervals such as 10, 18, 30, or 72 hours may also be employed depending on the clinical context, subject physiology, and diagnostic objectives. The sampling strategy is further enhanced by circadian alignment, early evening meal scheduling (e.g., between 6:00 PM and 8:00 PM), restriction of fluid intake during fasting windows, and precise documentation of sample collection timepoints. This structured approach enables detection of temporal shifts in biomarker expression, thereby improving the sensitivity, reproducibility, and predictive accuracy of the diagnostic platform.

In certain embodiments, biological samples such as urine, blood, saliva, and stool are collected in parallel or in sequence to enable computation of Reserve Excretion Ratios (RER™) for specific biomarkers. These may include, but are not limited to, phytochemicals (e.g., polyphenols), lipid peroxidation products (e.g., malondialdehyde [MDA], 4-hydroxynonenal [4-HNE]), and nucleic acid species (e.g., cfDNA, cfRNA, cf-mtDNA). This data enables the identification of subclinical shifts in redox balance, mitochondrial activity, detoxification efficiency, and inflammatory load.

Diagnostic interpretation is performed using a hybrid computational engine that integrates rule-based logic, machine learning algorithms, and quantum-inspired reasoning. This hybrid framework is capable of processing nonlinear, fuzzy, and multistate biological data to generate personalized diagnostic scores, disease risk alerts, and health trajectory predictions. By aligning biomarker sampling with circadian and metabolic states, the invention provides a dynamic, individualized, and preventive diagnostic solution that significantly outperforms conventional, static, and compartment-limited diagnostic systems.

### DETAILED DESCRIPTION OF THE INVENTION

The present invention introduces a comprehensive, compartment-specific sampling framework that integrates multiple biological sample types to assess both systemic molecular reserves and organ-specific excretion pathways. As used herein, the term “biological sample” refers to any material obtained from, associated with, or derived from a human or non-human subject whether in natural, altered, or simulated form that can provide diagnostically or prognostically relevant molecular information. The scope of “biological sample” is to be interpreted broadly and includes, without limitation, biological fluids such as blood, plasma, serum, urine, saliva, cerebrospinal fluid (CSF), lymph, amniotic fluid, synovial fluid, and interstitial fluid; tissue specimens including biopsy material, surgically resected tissue, autopsy-derived samples, cryopreserved tissues, and microdissected sections; and cellular materials such as isolated or enriched populations including primary cells, exfoliated cells, stem cells, immune cells, circulating tumor cells (CTCs), and extracellular vesicles like exosomes. As described in paragraph [0004], this flexible and integrative framework enables the invention to capture high-resolution molecular data across diverse physiological compartments and temporal states for early diagnosis, health monitoring, and therapeutic guidance.

### Multi-Biofluid Sampling for Diagnostic Assessment:

In one embodiment of the invention, the diagnostic system incorporates a diverse range of biological sample types to facilitate multi-compartment molecular profiling. Sample types include, but are not limited to, blood, which reflects systemic molecular reserves and provides insights into organelle-specific markers such as cell-free mitochondrial DNA (cf-mtDNA), nuclear cfDNA, cfRNA, immune mediators including cytokines, and broader metabolic trends. Urine serves as a primary excretory fluid and is used to assess detoxification and elimination efficiency by enabling measurement of polyphenols, oxidative byproducts such as malondialdehyde (MDA), cell-free nucleic acids, and metabolic waste compounds. Saliva, sputum, and dental biofilm function as non-invasive matrices that reflect mucosal immunity, oxidative stress burden, and oral-systemic microbial dynamics. Stool provides comprehensive insights into gut microbiome composition, digestion-derived metabolites, inflammatory biomarkers, and microbial DNA and RNA signatures relevant to gastrointestinal and systemic health. Other biological fluids and surface-accessible materials, such as sweat, tears, earwax, hair, scalp swabs, skin microbiota, wound exudates, cerebrospinal fluid (CSF), bronchoalveolar lavage fluid, amniotic fluid, placental tissue, and similar matrices, offer valuable diagnostic data related to neural, dermal, respiratory, immune, or fetal-maternal physiology. Reproductive fluids, including semen, menstrual effluent, cervical mucus, and vaginal secretions, provide insights into hormonal regulation, fertility parameters, mucosal immunity, and microbiome composition specific to the reproductive system. This multiplex, multi-biofluid sampling framework supports both reserve-based analysis, such as quantifying molecular retention in systemic compartments, and excretion-based analysis, such as evaluating clearance through urine, stool, and sweat. By enabling early detection of functional imbalances, organ-specific stress, and subclinical disease trends, the sampling system forms a foundational component of the present invention. Furthermore, the sampling approach is adaptable to both invasive and non-invasive collection methods and is designed to accommodate time-sensitive protocols aligned with fasting cycles, metabolic transitions, circadian rhythms, and physiological or environmental modulation.

Nucleic Acid Profiling (Intact, Fragmented, and Modified Forms): As used herein, the term “nucleic acid” refers to any polymeric molecule composed of nucleotide sequences that carry, regulate, or signal genetic, transcriptomic, epigenetic, or synthetic biological information. The term includes but is not limited to: Genomic DNA (gDNA): Nuclear-encoded DNA comprising the complete genome. Plasmid DNA: Circular, extrachromosomal DNA, whether natural or engineered. Mitochondrial DNA (mtDNA): Organelle-specific DNA used as a marker for mitochondrial function, oxidative stress, and aging. Cell-Free DNA (cfDNA): Fragmented extracellular DNA detected in fluids such as plasma, urine, saliva, and CSF, and further subclassified into: cf-mtDNA (mitochondrial origin), cf-nDNA (nuclear origin). cf-cytDNA (cytoplasmic) and cf-exosomal DNA (vesicle-enclosed). Messenger RNA (mRNA): Protein-coding RNA transcripts, either intracellular or circulating in biofluids. Cell-Free RNA (cfRNA): Includes free-floating or vesicle-encapsulated RNA from tissues or organ systems, encompassing: MicroRNAs (miRNA), Long non-coding RNAs (lncRNA) and Circular RNAs (circRNA). Synthetic Nucleic Acids and Oligonucleotides: Including chemically modified molecules such as Locked Nucleic Acids (LNA), Peptide Nucleic Acids (PNA), phosphorothioate-linked oligos, antisense strands, aptamers, and molecular barcodes. Mutated or Oxidized DNA: e.g., 8-hydroxy-2′-deoxyguanosine (8-OHdG), which indicates oxidative DNA damage or disease-related mutations. Epigenetic Modifications: Such as DNA methylation patterns, histone acetylation/methylation marks, and chromatin immunoprecipitation (ChIP)-identified structural changes. These nucleic acid species may be isolated in intact or fragmented forms and serve as key diagnostic and prognostic biomarkers. Their presence, concentration, fragmentation profile, sequence alteration, methylation status, or redox damage can indicate cellular stress, immune activation, organelle dysfunction, microbial dysbiosis, or systemic disease progression.

Phytochemical and Antioxidant Signature: As used herein, the term “phytochemical” refers to any plant-derived, fungal, algal, herbal, or synthetically derived bioactive compound capable of exerting antioxidant, detoxifying, immunomodulatory, anti-inflammatory, metabolic-regulatory, or other physiologically relevant effects. Such compounds include, without limitation, chemical classes comprising polyphenols, flavonoids, terpenoids, alkaloids, curcuminoids, stilbenes, lignans, tannins, glucosinolates, and any functional analogs, derivatives, or isomers thereof. As used herein, the term “antioxidant” includes both endogenous and exogenous molecules whether enzymatic or non-enzymatic that act to reduce or neutralize reactive oxygen species (ROS), reactive nitrogen species (RNS), or other oxidative agents. Antioxidants include, without limitation: Enzymatic antioxidants, such as superoxide dismutase, catalase, and glutathione peroxidase. Non-enzymatic antioxidants, such as glutathione, uric acid, ascorbic acid (vitamin C), and tocopherols (vitamin E). Phytochemical antioxidants, such as those derived from polyphenolic structures. As used herein, “polyphenol” is a subclass of phytochemicals characterized by one or more phenolic structural units and includes, by way of example and not limitation, quercetin, catechins (e.g., EGCG), curcumin, rutin, gallic acid, resveratrol, ellagic acid, ferulic acid, tannic acid, and functional equivalents thereof. Such phytochemicals and antioxidants may be detected in systemic, excretory, and surface-accessible biological compartments, including but not limited to blood, plasma, serum, urine, saliva, sweat, stool, reproductive fluids, wound exudates, breast milk, and skin surface samples. The relative levels and distribution of such compounds are herein utilized to compute diagnostic indices including, without limitation: REDOX-R™ (Redox Reserve Ratio): indicative of retained systemic antioxidant status. ANTIOX-EX-R™ (Antioxidant Excretion Ratio): indicative of antioxidant clearance via excretory routes. PHYTO-R™ (Phytochemical Reserve Index): indicative of net phytochemical retention and biological uptake. These indices, alone or in combination with other biomarkers disclosed herein, are applied to detect, score, and predict oxidative stress burden, dietary phytochemical sufficiency, detoxification efficiency, and redox-related physiological imbalances.

Oxidative Stress Biomarker Signature: As used herein, the term “oxidative stress biomarker” refers to any measurable molecule, adduct, or degradation product that reflects the presence, accumulation, or biological impact of oxidative processes involving lipids, proteins, nucleic acids, or other cellular components. Representative biomarkers include, but are not limited to: Lipid peroxidation products, such as malondialdehyde (MDA), 4-hydroxynonenal (4-HNE), isoprostanes, and acrolein. Protein oxidation markers, including carbonylated proteins and their conjugates. Nucleic acidfHAQ oxidation adducts, such as MDA-DNA complexes, 4-HNE-RNA adducts, and 8-hydroxy-2′-deoxyguanosine (8-OHdG). These oxidative markers may be evaluated across one or more biological compartments described herein, including systemic and excretory fluids, and may be used in combination with antioxidant and phytochemical profiles to derive a composite Oxidative Stress Score, herein defined as a numerical or categorical indicator of redox imbalance, cellular damage, and oxidative load across the organism. Such integration of oxidative and antioxidant profiles is intended to provide early warning, trend analysis, or therapeutic guidance for disease risk, metabolic dysfunction, environmental toxin exposure, or immune activation, as more fully described elsewhere herein.

Immune and Hormonal Mediators: As used herein, the term “immune and hormonal mediators” encompasses any molecule, compound, factor, or signal—whether protein-based, peptide-based, lipid-based, glycosylated, or otherwise—that modulates, reflects, or influences physiological processes related to immunity, inflammation, endocrine regulation, metabolic control, homeostatic adaptation, or circadian rhythm. This term broadly includes, without limitation: Cytokines, such as interleukins, tumor necrosis factors, interferons, chemokines, and colony-stimulating factors, including but not limited to IL-1β, IL-6, IL-10, TNF-α, IFN-γ, and any functionally equivalent or structurally related cytokine that modulates inflammatory or immune responses. Growth factors, such as angiogenic, mitogenic, or tissue-regenerating signals, including vascular endothelial growth factor (VEGF) and epidermal growth factor (EGF), and their biologically active isoforms, fragments, or mimetics. Hormones, whether steroidal, peptide, or amino-acid-derived, are secreted by endocrine organs or peripheral tissues and contribute to systemic regulation. This includes, but is not limited to, insulin, cortisol, estrogen, testosterone, and any analogs, precursors, or receptor-binding ligands. Organ-specific enzymes that act as surrogate markers of tissue integrity, functional status, or organ stress, such as ALT, AST, amylase, and equivalents, derivatives, or isoenzymes. Circadian and stress-related modulators, including neuroendocrine and metabolic signals such as melatonin and leptin, as well as any molecule implicated in sleep-wake cycling, appetite regulation, or adaptive stress response. The mediators described herein may be present in any biological matrix-systemic, excretory, or compartmentalized-including but not limited to blood, plasma, serum, urine, saliva, sweat, stool, cerebrospinal fluid, reproductive fluids, or tissue secretions. Measurement, classification, and trend analysis of these mediators may be performed alone or in conjunction with other molecular features described herein, such as oxidative stress markers, nucleic acids, phytochemicals, or diagnostic ratios. Their levels, ratios, and temporal fluctuations form part of the broader integrated diagnostic and prognostic system disclosed in this specification.

Microbial and Parasitic Nucleic Acids: As used herein, the term “microbial and parasitic nucleic acids” refers to DNA, RNA, or structurally related nucleic acid derivatives of microbial or parasitic origin, whether naturally occurring or synthetically modified. These may include, without limitation, cell-free microbial DNA (cfDNA), microbial RNA (cfRNA), plasmids, episomes, replicative intermediates, and nucleic acid fragments derived from: Bacteria, Viruses, Fungi, Archaea, Protozoa, Helminths and Other known or emerging microorganisms capable of colonizing or translocating within the host Such nucleic acids may originate from mucosal barrier sites or tissue compartments including the gastrointestinal tract, oral cavity, respiratory system, reproductive and urinary tracts, dermal surfaces, ocular membranes, and nasal passages. Once released into the host environment, they may circulate freely or be bound to extracellular vesicles, immune complexes, or protein carriers. These microbial nucleic acids can be present in systemic and excretory fluids such as blood, urine, saliva, stool, or cerebrospinal fluid. The presence, abundance, mutation profile, or structural form of these molecules may provide diagnostic insight into: Mucosal barrier disruption (e.g., intestinal or epithelial leakage), Microbial translocation and systemic inflammation, Dysbiosis, occult infections, or microbiome imbalance and Host-microbe interaction and immune modulation. These nucleic acids may be analyzed independently or in combination with other molecular features disclosed herein, including cfDNA/cfRNA, oxidative stress markers, mitochondrial signals, and immune mediators. They form a foundational component of the invention's integrated platform for evaluating microbial burden, inflammatory activation, and tissue-specific health status.

Metabolites and Toxins: As used herein, the term “metabolites and toxins” encompasses any endogenous or exogenous biochemical compound, whether known or yet to be identified, that reflects physiological, metabolic, detoxification, or organ-specific function in a subject. This includes, without limitation, naturally occurring small molecules such as glucose, lactate, creatinine, and uric acid, as well as bioaccumulated or pathogenic compounds such as heavy metals, uremic toxins, aflatoxins, volatile organic compounds, xenobiotics, and any metabolic byproduct or reactive intermediate relevant to health assessment. The term includes structurally or functionally analogous molecules, modified forms, derivatives, conjugates, isotopically labeled variants, and synthetic mimics of these metabolites and toxins. These analytes may be detected in systemic compartments (e.g., blood, plasma, serum) and excretory fluids (e.g., urine, saliva, sweat, stool), across dynamic physiological states including, but not limited to, feeding, fasting, inflammation, exertion, circadian rhythm, or recovery.

The scope of analysis herein is not limited by the origin, chemical class, detection method, or biological matrix of these metabolites and toxins. Rather, they are interpreted as part of a temporally stratified, quantum-informed diagnostic framework that integrates their presence, concentration, inter-compartmental ratio, and clearance kinetics with other biomolecular layers including cfDNA, cfRNA, immune mediators, oxidative stress indicators, phytochemicals, mitochondrial nucleic acids, and microbial DNA/RNA. The inclusion of such data into composite indices-generated via hybrid algorithmic models-supports predictive diagnostics, systemic redox profiling, detoxification efficiency scoring, and disease risk stratification. This enables personalized health management and supports the evolution of future claims involving novel metabolite panels, emerging environmental toxins, or stress-related metabolic profiles.

Hybrid Algorithmic-Quantum Logic Interpretation Framework (HAQ Diagnostic Index): The present invention introduces the HAQ Diagnostic Index™, a proprietary hybrid computational framework that integrates conventional algorithmic methods with quantum logic simulation to interpret complex and probabilistic biological data. Unlike existing systems that rely solely on binary or rule-based logic, the HAQ Diagnostic Index blends deterministic approaches such as machine learning (ML), statistical modeling, and decision trees with quantum-informed logic to accommodate physiological variability, uncertainty, and multistate transitions inherent in living systems. This interpretive engine is configured to process data obtained from multi-biofluid sampling, including but not limited to cell-free DNA (cfDNA), cell-free RNA (cfRNA), oxidative stress markers (e.g., malondialdehyde [MDA], 4-HNE), immune mediators (e.g., cytokines), microbial nucleic acids, dietary phytochemicals (e.g., polyphenols), metabolic toxins, and hormonal indicators. The system calculates critical indices such as the Reserve-Excretion Ratio (RER™), Quantum Diagnostic Score (QDS), Antioxidant Indices (e.g., REDOX-R™, ANTIOX-EX-R™), and Predictive Aging Index, all derived from spatial and temporal biomarker dynamics across fasting states and metabolic cycles. The HAQ Diagnostic Index models dynamic physiological states including but not limited to stress, inflammation, fasting, circadian rhythm, and emotional fluctuations. It integrates threshold-based outputs with probabilistic inference and fuzzy set theory to detect subtle, subclinical patterns that may precede overt disease. This allows for the generation of predictive, personalized diagnostics, prognostics, and therapeutic recommendations that are context-aware, adaptable to diverse demographic profiles, and resilient across varied health statuses. The system is modular, allowing integration of new biomarkers, sample types, and lifestyle/environmental data without structural overhaul. It is designed for continuous learning through recursive training on population-level datasets and longitudinal individual health profiles. Furthermore, it supports secure, real-time deployment across clinical, remote, and home-based platforms, utilizing cloud or edge-computing infrastructure. The term “quantum logic” as used herein encompasses, but is not limited to, non-classical logic techniques such as state superposition mapping, entanglement-informed decision paths, probabilistic coherence modeling, and multivalued truth functions.

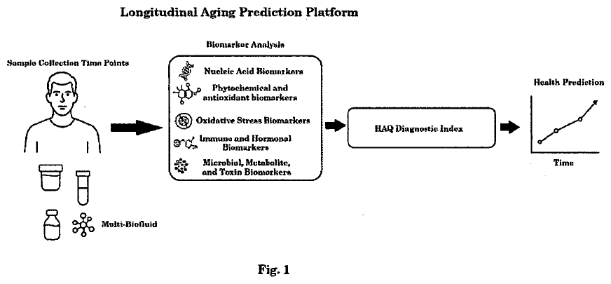

FIG. 1 illustrates this hybrid framework visually, showing how multi-biofluid sample inputs are processed through biomarker analysis modules and interpreted via the HAQ Diagnostic Index™ to produce a time-based health prediction curve for longitudinal aging assessment.

Diagnostic Framework Based on HAQ Diagnostic Index™: The HAQ Diagnostic Index™ functions as the central computational and interpretive engine of the disclosed multilayer diagnostic platform. It is configured to assess individual health status, predict disease progression, and guide personalized therapeutic recommendations. The system integrates and analyzes a comprehensive set of biomarkers and contextual parameters, including but not limited to the following categories.
Molecular Biomarkers: cf-mtDNA, cfRNA, inflammation-linked RNA transcripts, oxidative stress markers (MDA, 4-HNE), and epigenetic modifications (e.g., DNA methylation, 8-OHdG).
Phytochemical and Antioxidant Profiles: Total urinary polyphenols and dietary antioxidants such as quercetin, hesperidin, resveratrol, and rutin.
Microbiome Composition: Functional and compositional analysis of gut, oral, skin, and urogenital microbiota.
Immune and Hormonal Mediators: Cytokines including IL-6, IL-10, TNF-α, along with metabolic hormones.
Environmental and Lifestyle Stressors: Dietary components, toxins, circadian misalignment, emotional stress, fasting status, and physical activity.

### HAQ Index Output and Predictive Utility:

The output of the HAQ Diagnostic Index™ includes a predictive scoring system that evaluates:
Likelihood of disease onset, progression, or remission.
Individual responsiveness to medical or lifestyle-based interventions.
Relapse risk or chronicity likelihood.
Systemic resilience, redox efficiency, and adaptive physiological response.

### Modular System Design:

The HAQ system is designed for scalability and legal robustness. Its architecture accommodates future integration of novel biomarkers and diagnostic features, while its hybrid logic approach ensures broad patent coverage and resistance to algorithmic imitation. It also supports comparative biological models (e.g., Peddamma-logical frameworks) for cross-species reference.

### Diagnostic Consultation Framework Using HAQ Diagnostic Index:

The invention discloses a structured, five-stage diagnostic consultation framework configured to operate in conjunction with the HAQ Diagnostic Index™. This model enables integrated evaluation across physical, psychological, molecular, and environmental dimensions to support individualized diagnostic interpretation, risk stratification, and therapeutic decision-making. The stages include, but are not limited to, the following categories.

Stage 1: Five-Level Interview Evaluates the patient across:
Physical Level: Symptomatology and functional limitations.
Emotional Level: Stressors, anxiety, coping capacity.
Intellectual Level: Duration, triggers, pattern recognition.
Self-Awareness Level: Lifestyle patterns, behavior.
Diagnostic Readiness: Perception and preparedness for testing.

Stage 2: Diagnostic Testing
Macro-Level: Visual assessment of samples (urine, stool, saliva, skin, nails)
Micro-Level: Quantitative analysis of cfDNA, cfRNA, MDA, cytokines, microbiome, and polyphenol levels

Stage 3: HAQ Decision Matrix Integrates interview and biomarker data to classify health status into:
Reversible
Partially Reversible
Degenerative

Stage 4: Personalized Treatment Layers Tailors interventions across each level:
Physical: Nutraceuticals, detox protocols.
Emotional: Mindfulness, breathing, counseling.
Intellectual: Health education, behavioral tracking.
Self-Awareness: Diet correction, circadian alignment.
Diagnostic: Supplementation, immune modulation.

Stage 5: Prognosis via HAQ Score Longitudinal monitoring of HAQ scores enables real-time tracking of recovery, progression, or relapses, informing future care plans and optimizing health resilience.

Experimental Validation of the Invention: The present invention was experimentally validated using biological samples collected from both healthy volunteers and clinically diagnosed individuals representing a range of conditions including metabolic, mitochondrial, inflammatory, and infectious disorders. Validation was performed using the novel time-staggered, multi-fluid, fasting-based protocol involving sample collections at 12-hour (overnight), 24-hour (prolonged), and 36-hour (extended, physician-supervised) fasting intervals. Sample matrices included urine, blood, saliva, and stool.

The samples were analyzed for multiple biomolecular classes: Cell-free nucleic acids (cf-mtDNA, cf-nDNA, cfRNA, miRNA). Polyphenols (e.g., quercetin, rutin, hesperidin, resveratrol). Oxidative stress biomarkers (e.g., MDA, 4-HNE, protein carbonyls). Epigenetic markers (e.g., 8-OHdG, DNA methylation). Microbial/parasitic DNA. Immune mediators (cytokines, interleukins, hormones). Metabolites and toxins (e.g., lactate, heavy metals, aflatoxins).

Two extraction methods for cf-mtDNA were compared: Physical separation (centrifugation-based) and Chemical phase-separation (proprietary method).

Physically separated cf-mtDNA failed to distinguish normal vs. abnormal cases consistently, often yielding overlapping copy numbers and false negatives.

Chemically separated cf-mtDNA showed clear stratification. Abnormal patients consistently exhibited elevated copy numbers (above 15,000), while normal individuals remained under this threshold (except for subclinical case P5).

Supporting tests: Total urinary polyphenol concentration and Urinary MDA as an oxidative stress marker.

Data revealed that asymptomatic patients with abnormal cf-mtDNA profiles also showed elevated MDA and polyphenol levels, confirming subclinical molecular stress.

Multiphasic analysis using the hybrid algorithm confirmed these cases as molecularly abnormal, validating early disease detection potential.

### Summary of Data Tables:

TABLE 1
Physically Separated cf-mtDNA from Urine.

| | Patient | cf-mtDNA Copy Number |
|---|---|---|
| Name | Status | (copy no/mL) |
| P1 | Normal | 1984 |
| P2 | Normal | 716 |
| P3 | Normal | 317 |
| P4 | Normal | 1116 |
| P5 | Normal | 624 |
| P6 | Abnormal | 3555 |
| P7 | Abnormal | 38610 |
| P8 | Abnormal | 7692 |
| P9 | Abnormal | 984 |

TABLE 2
Chemically Separated cf-mtDNA from Urine.

| | Patient | cf-mtDNA Copy Number |
|---|---|---|
| Name | Status | (copy no/mL) |
| P1 | Normal | 4342 |
| P2 | Normal | 207 |
| P3 | Normal | 1019 |
| P4 | Normal | 1259 |
| P5 | Normal | 16541 |
| P6 | Abnormal | 30420 |
| P7 | Abnormal | 39960 |
| P8 | Abnormal | 31450 |
| P9 | Abnormal | 21560 |

TABLE 3
Total Polyphenols from Urine.

| | Patient | Total Polyphenols |
|---|---|---|
| Name | Status | (μmol/L) |
| P1 | Normal | 3662 |
| P2 | Normal | 3521 |
| P3 | Normal | 2547 |
| P4 | Normal | 2354 |
| P5 | Normal | 6235 |
| P6 | Abnormal | 7800 |
| P7 | Abnormal | 8541 |
| P8 | Abnormal | 9102 |
| P9 | Abnormal | 8769 |

TABLE 4
MDA from Urine.

| Name | Patient Status | MDA (mg/L) |
|---|---|---|
| P1 | Normal | 2.1 |
| P2 | Normal | 1.2 |
| P3 | Normal | 3.1 |
| P4 | Normal | 1.7 |
| P5 | Normal | 6.9 |
| P6 | Abnormal | 7.8 |
| P7 | Abnormal | 6.4 |
| P8 | Abnormal | 7.6 |
| P9 | Abnormal | 7.1 |

The present invention was experimentally validated through comparative testing of urine samples collected from both clinically normal and abnormal patients. The core objective was to evaluate the reliability and diagnostic power of cell-free mitochondrial DNA (cf-mtDNA) levels when extracted using two distinct methods.

Physical Separation Method: This approach employed traditional centrifugation-based techniques to isolate cell-free fractions from urine samples. The resulting cf-mtDNA copy numbers from both patient groups are presented in Table 1.

A chemically optimized phase-separation technique was employed for the extraction of biomolecular targets, including, but not limited to, cell-free mitochondrial DNA (cf-mtDNA), from the same set of urine samples. This chemical method was systematically benchmarked against conventional physical separation techniques, including, but not limited to, centrifugation, membrane-based filtration materials (such as spin columns, nitrocellulose membranes, or polymeric filters), and gravity-induced settling. The results, as summarized in Table 2, demonstrate that the chemically optimized method achieved superior diagnostic resolution and consistent biomarker stratification between clinically normal and abnormal individuals. These findings validate the enhanced sensitivity, reproducibility, and predictive utility of the chemical extraction method over standard physical separation techniques

The physically separated cf-mtDNA values demonstrated significant overlap between normal and abnormal patients. As shown in Table 1, samples P6 (Abnormal, 3555 copies), P9 (Abnormal, 984), and others exhibited values that were comparable to or even lower than those from clinically normal individuals, such as P1 (Normal, 1984) and P4 (1116). This overlap resulted in false-negative interpretations, reducing the diagnostic sensitivity of the physical separation approach.

In contrast, chemically separated cf-mtDNA data exhibited a clear stratification between normal and abnormal patients, as presented in Table 2. All abnormal patients (P6-P9) demonstrated cf-mtDNA copy numbers well above 15,000 while most normal samples remained below this threshold. One exception, P5 (Normal), exhibited elevated cf-mtDNA (16,541), suggesting possible subclinical or asymptomatic molecular stress.

To further investigate these discrepancies, especially among patients with no outward symptoms, two additional biochemical markers were evaluated: Total Polyphenols in Urine (see Table 3). Malondialdehyde (MDA) levels, a marker of oxidative stress (see Table 4)

Notably, P5 (Normal) exhibited elevated polyphenol levels (6235) and MDA (6.9)—values comparable to those observed in abnormal patients. This confirms the diagnostic advantage of the chemical extraction method when combined with oxidative and phytochemical profiling.

A multiphasic analysis integrating cf-mtDNA, total polyphenols, and MDA revealed that multiple clinically normal individuals, particularly P5, displayed molecular-level anomalies. When analyzed using the proposed hybrid algorithm and quantum-level diagnostic scoring system, these patients were flagged as ‘abnormal’, despite lacking physical symptoms.

The layered validation using physical, chemical, and biochemical markers underscores the robustness and predictive capacity of the diagnostic system. This approach enables early detection of pathological changes, well before the manifestation of clinical symptoms, and supports the invention's utility in preventive, personalized healthcare.

Conclusion with Data-Backed Justification. The data presented in Table 1 (Physically Separated cf-mtDNA) and Table 2 (Chemically Separated cf-mtDNA) provide compelling comparative evidence. While physical separation methods failed to clearly distinguish abnormal from normal samples-evidenced by overlapping values such as P6 (3555) and P9 (984) appearing within the normal range-chemical separation produced consistently elevated cf-mtDNA levels in abnormal patients (e.g., P6: 30,420; P7: 39,960; P8: 31,450); P9: 31,560), while normal individuals largely remained under 17,000, except P5 (16,541), which is discussed below.

Chemically separated cf-mtDNA extraction is more sensitive and clinically predictive than physical separation.

Multiphasic analysis of cf-mtDNA, MDA, and Total Polyphenols reveals early disease trends even in physically normal individuals.

Extended Validation: Phytochemical and Oxidative Stress Profiling of Patient P5. In addition to the primary cf-mtDNA comparison study, Patient P5 originally classified as clinically normal-underwent further analysis to assess biomolecular reserve and excretion patterns. This extended evaluation focused on antioxidant polyphenols and the lipid peroxidation marker malondialdehyde (MDA), measured in both blood and urine compartments.

For each target molecule, two diagnostic ratios were computed: Urine PP/Blood PP: indicating body excretion rate and Blood PP/Urine PP: indicating systemic retention or reserve. The measured values for Patient P5 are presented in Table 5.

TABLE 5
Body Reserve and Excretion Ratios for
Polyphenols and MDA - Patient P5

| Target | Urine PP/Blood PP | Blood PP/Urine PP |
|---|---|---|
| Total Polyphenols | 2.4 | 0.4 |
| Hesperidin | 1.6 | 0.6 |
| Rutin | 2.8 | 0.4 |
| Quercetin | 0.4 | 2.7 |
| Resveratrol | 6.9 | 0.1 |
| Ellagic Acid | 2.7 | 0.4 |

| Target | Urine MDA/Blood MDA | Blood MDA/Urine MDA |
|---|---|---|
| MDA | 5.3 | 0.2 |

These results indicate a diagnostic imbalance. While quercetin exhibited good systemic retention (2.7), other phytochemicals such as resveratrol (6.9), rutin (2.8), and ellagic acid (2.7) showed significantly higher excretion than reserve, suggesting excessive antioxidant loss or underutilization.

The MDA ratio (5.3 excretion vs. 0.2 reserve) reflects active oxidative clearance, implying systemic oxidative stress not evident through physical symptoms alone.

When this data was processed using the invention's hybrid diagnostic engine incorporating quantum-informed interpretation and biomarker ratio scoring Patient P5 was flagged as molecularly abnormal despite a clinically normal status.

This case illustrates the utility of integrated reserve-excretion diagnostics. The combined interpretation of cf-mtDNA levels, polyphenol ratios, and MDA trends enables early identification of hidden oxidative or metabolic stress. It validates the invention's predictive capabilities in identifying subclinical disease risk prior to traditional symptom onset.

Comparative RER™ Analysis: Normal vs. Fasting Abnormal Patient Profiles: To further validate the utility of the trade-named diagnostic index RER™ (Redox Reserve-Excretion Ratio), comparative testing was conducted between a clinically normal patient and a patient identified as abnormal under fasting conditions. Biomarkers analyzed included major dietary phytochemicals and the oxidative stress marker malondialdehyde (MDA), measured in both blood (reserve) and urine (excretion).

### Application of RER™ Ratios to Clinical Samples

The RER™ values are calculated as Blood PP/Urine PP, which indicates how well the body retains a compound in circulation relative to what it eliminates. A high RER™ indicates antioxidant sufficiency or retention; a low RER™ suggests depletion, overflow, or loss of metabolic control.

TABLE 6A
Normal Patient - RER ™ Biomarker Ratios.

| Target | Urine PP/Blood PP | Blood PP/Urine PP |
|---|---|---|
| Total Polyphenols | 0.3 | 3.0 |
| Hesperidin | 0.9 | 1.1 |
| Rutin | 0.0 | 77.9 |
| Quercetin | 0.2 | 4.1 |
| Resveratrol | 0.7 | 1.4 |
| Ellagic Acid | 0.0 | 44.3 |

| Target | Urine MDA/Blood MDA | Blood MDA/Urine MDA |
|---|---|---|
| MDA | 0.3 | 3.1 |

TABLE 6B
Fasting Abnormal Patient - RER ™ Biomarker Ratios.

| Target | Urine PP/Blood PP | Blood PP/Urine PP |
|---|---|---|
| Total Polyphenols | 2.3 | 0.4 |
| Hesperidin | 12.6 | 0.1 |
| Rutin | 0.5 | 2.2 |
| Quercetin | 3.9 | 0.3 |
| Resveratrol | 6.6 | 0.2 |
| Ellagic Acid | 0.2 | 5.4 |

| Target | Urine MDA/Blood MDA | Blood MDA/Urine MDA |
|---|---|---|
| MDA | 3.7 | 0.3 |

Diagnostic Interpretation Using RER™: The normal patient's RER™ values (e.g., 4.1 for quercetin, 3.0 for total polyphenols, 3.1 for MDA) indicate robust antioxidant retention and efficient redox homeostasis. In contrast, the fasting abnormal patient shows very low RER™ values for most targets (e.g., 0.1 for hesperidin, 0.2 for resveratrol, 0.3 for MDA), reflecting oxidative overload and poor antioxidant buffering capacity-despite the absence of clinical symptoms.

Diagnostic Integration via Hybrid Algorithm: Rule-based thresholds for biomarker cut-offs. Machine learning to detect multi-biomarker trends. Quantum-informed models for nonlinear feedback detection.

TABLE 7
The system outputs a composite Quantum Diagnostic Score (QDS)
derived from RER ™ indices and time-point dynamic data, enabling personalized
trend tracking and early risk stratification.

| | | cf-mtDNA | | | cf-mtDNA/ | Polyphenol/ | |
|---|---|---|---|---|---|---|---|
| | Patient | (copy | Polyphenol | MDA | MDA | MDA | HAQ Diagnostic |
| Name | Status | no/mL) | (μmol/L) | (mg/L) | Ratio | Ratio | Index |
| P1 | Normal | 4342 | 3662 | 2.1 | 2068 | 1744 | 1938 |
| P2 | Normal | 207 | 3521 | 1.2 | 173 | 2934 | 1277 |
| P3 | Normal | 1019 | 2547 | 3.1 | 329 | 822 | 526 |
| P4 | Normal | 1259 | 2354 | 1.7 | 741 | 1385 | 998 |
| P5 | Normal | 16541 | 6235 | 6.9 | 2397 | 904 | 1800 |
| P6 | Abnormal | 30420 | 7800 | 7.8 | 3900 | 1000 | 2740 |
| P7 | Abnormal | 39960 | 8541 | 6.4 | 6244 | 1335 | 4280 |
| P8 | Abnormal | 31450 | 9102 | 7.6 | 4138 | 1198 | 2962 |
| P9 | Abnormal | 21560 | 8769 | 7.1 | 3037 | 1235 | 2316 |

The HAQ Diagnostic Index helps to understand a person's internal health status based on key biological markers. A score between 0 and 800 means the person is healthy, and no action is needed. Scores from 801 to 1800 suggest early or hidden stress, and it's good to monitor regularly and make lifestyle improvements like better diet, sleep, or exercise. If the score is between 1801 and 3000, it indicates noticeable stress in the body, and a preventive health check or further testing is recommended. A score above 3000 is considered high risk, meaning the body is under serious stress, and medical attention along with recovery plans should be started as soon as possible.

Conclusion: Chemically The experimental validation of the present invention demonstrates its robust diagnostic utility and predictive sensitivity across a broad range of biological markers and health states. The comparative analysis between physically and chemically separated cf-mtDNA unequivocally establishes the superiority of the chemically optimized phase-separation technique in distinguishing clinically abnormal individuals from normal counterparts. The chemical method yielded cf-mtDNA copy numbers with consistent stratification, whereas the physical method showed overlapping ranges and frequent false negatives, undermining diagnostic accuracy. Further validation using oxidative stress markers (e.g., MDA) and dietary polyphenols confirmed the reliability of biomarker-based profiling in identifying subclinical physiological stress. Notably, patient P5, classified clinically as normal, exhibited elevated molecular markers and was successfully flagged as at-risk by the hybrid algorithm-underscoring the early detection capabilities of the system. The introduction of the Reserve-Excretion Ratio (RER™) and its application to polyphenols and MDA provided an advanced metric to assess redox imbalance and antioxidant reserve. Patients with low RER™ values displayed poor molecular retention and high excretion, consistent with systemic oxidative stress and functional imbalance, even in the absence of clinical symptoms. The hybrid algorithmic-quantum logic interpretation engine (HAQ Diagnostic Index™) effectively integrated these multidimensional data layers—biochemical, molecular, temporal—to generate personalized, actionable health scores. The system successfully stratified patients into risk categories ranging from normal (0-800) to high-risk (>3000), enabling preventive and predictive healthcare interventions. In totality, the validation data substantiate the novelty, efficacy, and clinical relevance of the invention. The invention enables multi-biofluid, multi-marker diagnostics aligned with fasting-state physiology and incorporates advanced computational logic for early, personalized, and non-invasive health risk assessment. This establishes a significant advancement over existing diagnostic approaches and supports the invention's broad applicability across preventive, therapeutic, and longitudinal health monitoring domains.

Detailed Proof of Concept—Longitudinal Aging Prediction Platform (CASE #5) Subject Overview and Study Design To demonstrate the real-world predictive utility of the present invention, a longitudinal, time-resolved biomarker monitoring study was performed on a single subject (Case #5). This proof-of-concept case illustrates how the diagnostic platform can predict health trajectories over time through multi-biofluid, multi-marker analysis, interpreted using a hybrid quantum algorithmic engine.

Subject Profile Age: 26 years, Sex: Male. Sample Collection Plan Samples were collected at three timepoints—baseline (0 months), 6 months, and 12 months—under both fasting (12-hour overnight fast) and non-fasting conditions to compare biomarker variability across physiological states.

Biofluids Analyzed: Including but Not Limited to Urine: Cell-free mitochondrial DNA (cf-mtDNA), malondialdehyde (MDA), cell-free RNA (cfRNA). Blood: Polyphenols (quercetin, hesperidin, rutin), MDA, interleukin-6 (IL-6), cell-free nuclear DNA (cfDNA). Saliva: Total polyphenols. Stool: Microbial diversity index (Shannon, Simpson), IL-6.

Biomarker Units Biomarker values were reported in the following standard units unless otherwise noted: Polyphenols: μmol/L, MDA: mg/L, All other biomarkers (cf-mtDNA, cfRNA, IL-6, Microbial Diversity Index): copy number/Ml

TABLE 8
Biomarker Values Across Timepoints

| | | | Total | IL-6 | | Microbial |
|---|---|---|---|---|---|---|
| | cf-mtDNA | | Polyphenols | (Stool) | cfRNA (Urine) | Diversity |
| | (Urine) | MDA (Urine) | (Saliva) | (copy | (copy | Index(Stool) |
| Timepoint | (copy no/mL) | (mg/L) | (μmol/L) | no/mL) | no/mL) | (copyno/mL) |
| Baseline | 15.6 | 2.8 | 38 | 22 | 3.5 | 1.7 |
| 6 months | 10.3 | 2.1 | 51 | 14 | 2.8 | 2.4 |
| 12 months | 7.1 | 1.4 | 65 | 9 | 2.2 | 3.1 |

TABLE 9
Diagnostic Ratios and Index Values

| | | | RER | |
|---|---|---|---|---|
| | cf- | | (Reserve- | HAQ |
| | mtDNA/MDA | Polyphenol/MDA | Excretion | Diagnostic |
| Timepoint | Ratio | Ratio | Ratio) | Index |
| Baseline | 5.57 | 13.57 | 0.72 | 0.68 |
| 6 months | 4.9 | 24.28 | 0.84 | 0.75 |
| 12 months | 5.07 | 46.42 | 0.91 | 0.88 |

### Lifestyle Guidance Generated (Including but not Limited to)

Based on biomarker dynamics and algorithmic modeling, the platform generated lifestyle recommendations to support long-term redox balance and mitochondrial health:
Maintain a polyphenol-rich diet (e.g., quercetin, hesperidin, rutin)
Continue intermittent fasting and yoga/mindfulness practices
Monitor cf-mtDNA, MDA, and polyphenols every 6 months
Apply emotional stress-reduction techniques to minimize IL-6 elevations and improve immune balance

### Quantum-Simulated Predictive Modeling Insight

The hybrid diagnostic engine simulates multi-year health trajectories using a quantum-informed uncertainty framework. In this case, the HAQ Diagnostic Index improved steadily from 0.68 at baseline to 0.88 in 12 months. This trajectory places the individual in a regenerative trend cluster (ΔHAQ>0.15/year), which is associated with lower risk of degenerative aging diseases over the next 3-5 years. If maintained, this trend predicts high biological resilience.

Conclusion: This Case #5: Longitudinal Aging Prediction. The longitudinal analysis conducted on Subject Case #5 provides compelling validation for the predictive capabilities of the present invention. By integrating multi-biofluid sampling, temporal biomarker tracking, and hybrid quantum-algorithmic interpretation, the system was able to monitor subtle yet significant physiological shifts over a 12-month period. Across three timepoints, a consistent improvement in key molecular biomarkers was observed, including a reduction in oxidative stress (urinary MDA), enhanced antioxidant intake (salivary polyphenols), reduced inflammatory signaling (stool IL-6), and increased microbial diversity. The cf-mtDNA/MDA and polyphenol/MDA ratios progressively improved, aligning with the anticipated biological trend toward redox balance and mitochondrial stability. The HAQ Diagnostic Index™—which encapsulates these biomarker trends through algorithmic scoring and quantum logic modeling—rose from 0.68 to 0.88 over one year, reflecting enhanced biological resilience. This gain (>0.15/year) qualified the subject for a regenerative trajectory cluster, which, according to platform simulations, is associated with reduced risk of degenerative aging diseases over the next 3 to 5 years. Moreover, the diagnostic platform was able to generate actionable lifestyle guidance at each stage, personalized to the subject's evolving molecular profile. Recommendations included polyphenol-rich dietary intake, intermittent fasting, stress regulation practices, and biomarker retesting intervals—each aligned with dynamic system outputs. This case study illustrates the platform's ability to (a) detect preclinical biomarker shifts, (b) generate longitudinal health forecasts, and (c) deliver adaptive intervention strategies based on biologically relevant indices. It confirms the core utility of the invention for predictive aging diagnostics and supports its broader application in preventive healthcare, regenerative medicine, and personalized longevity monitoring.

1. A diagnostic system comprising a compartment-specific sampling framework configured to extract and analyze biomarkers from a plurality of sample types selected from the group consisting of fluids, tissues, cells, secretions, and engineered or simulated constructs; wherein said biomarkers comprise at least one of nucleic acids, oxidative stress markers, phytochemicals, immune mediators, microbial nucleic acids, or metabolic toxins; wherein the system performs multi-compartment and multi-timepoint profiling under physiological states including fasting, circadian-modulated, and emotionally influenced conditions; and wherein the system generates diagnostic indices using a five-layer interpretive model comprising physical, chemical, biological, mathematical, and philosophical domains.

2. An integrated diagnostic platform comprising a chemically optimized extraction method configured to isolate cell-free nucleic acids, including, but not limited to, cell-free mitochondrial DNA (cf-mtDNA), from one or more biofluids collected under a circadian-aligned fasting protocol at two or more temporally spaced timepoints selected from, but not limited to, 12 hours, 24 hours, 36 hours, 48 hours, or extended intervals over multiple days; wherein said chemically optimized extraction method is optionally combined with one or more physical separation techniques, including centrifugation, membrane-based filtration, or gravity-induced settling; wherein the platform computes Redox Reserve Excretion Ratios (RER™) and related indices by comparing biomarker concentrations across systemic and excretory compartments; and wherein biomarker dynamics are interpreted using a hybrid quantum-algorithmic framework to generate temporally stratified health scores for early detection of oxidative stress, mitochondrial dysfunction, immune dysregulation, metabolic overload, and disease susceptibility.

3. A diagnostic interpretation framework comprising a hybrid algorithmic-quantum logic engine configured to process biomarker data from multi-biofluid samples; wherein the engine integrates rule-based thresholds, machine learning algorithms, and quantum logic models to generate predictive scores and therapeutic guidance; and wherein the engine enables real-time temporal data analysis, adaptive learning from population datasets, and deployment on edge or cloud infrastructure.

4. The diagnostic system of claim 1, wherein the sample types include both invasive and non-invasive biological samples selected from the group consisting of blood, urine, saliva, stool, sweat, exhaled breath condensate, vaginal secretions, and cerebrospinal fluid.

5. The diagnostic system of claim 1, wherein the biomarker profiling is performed under at least two physiological states selected from the group consisting of fasting, fed, stressed, circadian-aligned, or emotionally modulated conditions.

6. The diagnostic system of claim 1, wherein the diagnostic indices include at least one of: a Redox Reserve Excretion Ratio (RER™), a Phytochemical Reserve Index (PHYTO-R™), or a Quantum Diagnostic Score (QDS).

7. The diagnostic system of claim 1, wherein the five-layer interpretive model integrates molecular data with behavioral and lifestyle parameters including diet, fasting compliance, sleep cycles, and emotional stress responses.

8. The integrated diagnostic platform of claim 2, wherein the chemically optimized extraction method comprises a multiphasic or cloudy colloidal buffer system configured to extract nucleic acids directly from urine and other excretory fluids.

9. The integrated diagnostic platform of claim 2, wherein said biofluids are collected using a circadian-aligned sampling protocol that includes an early dinner, restricted hydration, and sample time documentation.

10. The integrated diagnostic platform of claim 2, wherein the extracted cf-mtDNA is quantified using real-time PCR or digital PCR and stratified based on concentration thresholds indicative of mitochondrial stress.

11. The integrated diagnostic platform of claim 2, wherein the system computes the Reserve-Excretion Ratio (RER™) based on concentration ratios of polyphenols and MDA between blood and urine.

12. The integrated diagnostic platform of claim 2, wherein the extracted data is further validated using oxidative stress markers selected from malondialdehyde (MDA), 4-hydroxynonenal (4-HNE), or 8-OHdG.

13. The integrated diagnostic platform of claim 2, wherein the hybrid interpretation engine integrates biomarker trends across at least three timepoints to track recovery, relapse, or health trajectory.

14. The integrated diagnostic platform of claim 2, wherein cf-mtDNA values obtained through chemical separation provide superior patient stratification compared to physical extraction methods.

15. The integrated diagnostic platform of claim 2, wherein the platform provides longitudinal health trend simulations using quantum-modeled projections based on changes in HAQ Diagnostic Index scores.

16. The integrated diagnostic platform of claim 2, wherein said biomarker-derived scores are used to generate individualized lifestyle recommendations including antioxidant-rich diet, intermittent fasting, and emotional stress regulation.

17. The diagnostic interpretation framework of claim 3, wherein the hybrid quantum logic engine is configured to process temporally resolved data collected at fasting intervals selected from 12-hour, 24-hour, and 36-hour periods.

18. The diagnostic interpretation framework of claim 3, wherein the engine applies non-binary quantum logic operations including probabilistic coherence modeling, state superposition, and multivalued inference for detecting subclinical abnormalities.

19. The diagnostic interpretation framework of claim 3, wherein the machine learning model is trained using multiparametric inputs including cf-mtDNA, MDA, total polyphenols, cytokines, and microbial nucleic acid patterns.

20. The diagnostic interpretation framework of claim 3, wherein the output comprises a personalized health score categorized into zones indicating: normal (0-800), early stress (801-1800), high stress (1801-3000), or high-risk (>3000).
